(19) United States
(12) Patent Application Publication
(10) Pub. No.: US 2012/0295338 A1
(43) Pub. Date: Nov. 22, 2012

(54) MONITORING SYSTEMS FOR BIOMASS PROCESSING SYSTEMS

(76) Inventors: Paul Reep, Ojai, CA (US); Gavin Gray, Sun Valley, CA (US)

(21) Appl. No.: 13/475,803

(22) Filed: May 18, 2012

Related U.S. Application Data
(60) Provisional application No. 61/488,650, filed on May 20, 2011.

(51) Int. Cl. C12M 1/34; C12M 1/36

(52) U.S. Cl. 435/286.1; 435/287.1

(57) ABSTRACT

A sensor array is disclosed that includes a conduit having a lumen extending between an inlet and an outlet of the conduit. The sensor array can include a plurality of sensors coupled to the conduit. Each sensor can have a probe member that is disposed within the lumen. The sensors can be configured to measure at least one parameter of an aqueous medium containing a biomass.

### RELATED APPLICATIONS

This application claims the benefit of U.S. Provisional Application No. 61/488,650, filed May 20, 2011, entitled SYSTEMS AND METHODS FOR MONITORING AND CONTROLLING PROCESS CHEMISTRY ASSOCIATED WITH BIOMASS, GROWTH, OIL PRODUCT AND OIL SEPARATION IN AQUEOUS MEDIUMS, which is hereby incorporated herein by reference in its entirety.

### BACKGROUND

1. Field of the Invention

The present invention relates generally to the fields of energy and biology, and particularly to biomass growth and processing systems. Some aspects of the invention relate to a sensor array configured to measure parameters of an aqueous medium containing a biomass.

2. Background

Products which may be derived from biomass, such as the intracellular products of microorganisms, show promise as partial or full substitutes for fossil oil derivatives or other chemicals used in manufacturing products such as, inter alia, pharmaceuticals, cosmetics, nutraceuticals, other food products, industrial products, biofuels, synthetic oils, animal feed, fertilizers and so forth. However, for these substitutes to become viable, methods for both fostering the growth and development of the biomass and obtaining and processing usable bio-based products must be efficient and cost effective in order to be competitive with the refining costs associated with fossil oil derivatives. Current systems and methods used for harvesting bio-based products for use as fossil oil substitutes are laborious and may yield low net energy gains, rendering them unfeasible for today's alternative energy demands. Further, such methods can produce a significant carbon footprint, exacerbating global warming and other environmental issues. These methods, when further scaled up, produce an even greater efficiency loss, due to valuable intracellular component degradation, and require greater energy or chemical inputs than what is currently financially and/or environmentally feasible from a commercially viable biomass harvest.

Recovery of intracellular particulate substances or products from biomass sometimes requires disruption, lysing or fracturing of the cell membrane. Intracellular extraction methods can vary greatly depending on the type of organism involved, their desired internal component(s), and their purity levels. Optimized biomass growth can also be organism dependent, and can require varied inputs over the life cycle of the biomass. Accordingly, there is a need for systems and procedures that can be used to improve the development of biomass feedstocks and the refinement of such feedstocks. Such systems and procedures may assist to develop a spectrum of bio-based products that can be used as competitively-priced substitutes for fossil oils and fossil oil derivatives required for manufacturing processes and energy production.

### SUMMARY

The present invention has been developed in response to problems and needs in the art that have not yet been fully resolved by currently available systems and methods. Thus, these systems and methods are developed to measure parameters of an aqueous medium containing a biomass. When measured, these parameters can be used to improve biomass growths and harvesting processes in order to improve the refinement and production of biomass feedstocks and products derived therefrom.

In some embodiments, a sensor array is provided. The sensor array can include a conduit having a lumen extending between an inlet and an outlet of the conduit. The sensor array can include a plurality of sensors coupled to the conduit. Each sensor can have a probe member that is disposed within the lumen. The sensors can be configured to measure at least one parameter of an aqueous medium containing a biomass. Non-limiting examples of parameters of an aqueous medium include pH, oxidation reduction potential (ORP), total dissolved solids (TDS), water hardness, temperature, conductivity, salinity, chlorine concentration, carbon dioxide (CO₂) concentration, ammonia concentration, dissolved oxygen concentration, zeta potential, and/or algae cell density.

In some embodiments, a biomass processing system includes a vessel, a sensor array, and a controller. The vessel can be configured to retain an aqueous medium containing a biomass. The sensor array can be in fluid communication with the vessel. The sensor array can have a plurality of sensors, each with a probe member disposed within the vessel. Each of the sensors can be configured to measure at least one parameter of the aqueous medium in which a biomass may grow. The controller can be in electronic communication with the sensor array. The controller can be configured to adjust one or more processes of the biomass processing system in response to sensor data received from the sensor array.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 is a block diagram of a representative biomass processing system according to some embodiments.

FIG. 2 is a cross-section view of a conduit having a sensor array, according to some embodiments.

FIG. 3 is a cross-section view of another conduit having a sensor array, according to some embodiments.

FIG. 4A is a perspective view of a sensor array according to some embodiments.

FIG. 4B is a top view of the sensor array of FIG. 4A.

FIG. 5A is a flow diagram of a sensor loop of a fluid flow system, according to some embodiments.

FIG. 5B is a flow diagram of a sensor loop utilizing a bypass loop to generate fluid flow in a reverse direction, according to some embodiments.

FIG. 6 is a biomass processing system and SCADA system according to some embodiments.

### DETAILED DESCRIPTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope. For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).”

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

The present invention relates generally to the fields of energy and biology, and particularly to biomass growth and processing systems. Some aspects of the invention relate to a sensor array configured to measure parameters of an aqueous medium containing a biomass.

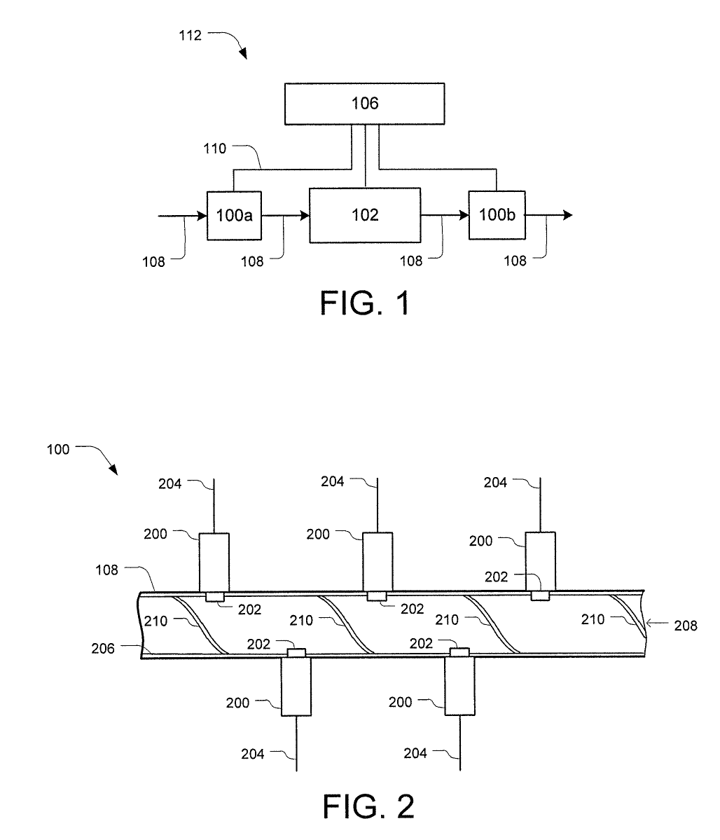

FIG. 1 illustrates a representative biomass processing system 112 in which a biomass feedstock culture, including, for example, microalgae, can be grown in an aqueous medium, harvested, and/or otherwise processed (herein “processed”). The illustrated biomass processing system 112 includes a conduit 108 or other vessel for containing or transporting the aqueous medium in a location or through a processing device 102 of the system. The location or processing device 102 can include growth tanks, growth ponds, bioreactors, biomass concentrators, lysing reactors, filter devices, heater devices, and other such locations and processing devices 102 of a biomass processing system 112.

In the representative system, two sensor arrays 100a and 100b (generally 100) are coupled to the conduit 108 and in fluid communication with the aqueous medium. The sensor array 100 can include a plurality of sensors. Each sensor can be configured to measure at least one parameter of the aqueous medium. The measured parameters can be key parameters in the growth and physiology of the biomass. The measurement and subsequent control of these parameters can be useful in optimizing the growth of a biomass feedstock as well as in harvesting the feedstock. For example, in the biomass growth stage, a sensor array 100 can measure water chemistry and algae culture parameters of the aqueous growth medium. The measured parameters can be analyzed to monitor biomass growth and determine if changes need to be made to the growth system, such as adding micronutrients. In another example, the sensor array 100 may be able to measure parameters that indicate when the feedstock is ready for harvesting. In yet another example, one or more sensor arrays 100 can measure water chemistry and algae culture parameters during algae harvesting to configure harvesting process systems.

While FIG. 1 illustrates two sensor arrays 100 in a biomass processing system 112, it will be understood that any number of sensor arrays 100 can be included in such systems. The number of sensor arrays 100 can depend on the size of the system, the throughput and volume of aqueous medium through a given system structure, and the number of system components that rely or benefit from the measurements provided by the sensor array 100. Moreover, some biomass processing systems 112, particularly growth systems or growth vessel may only need as single sensor array 100 to measure the parameters of the aqueous growth medium.

A sensor array 100 can include a plurality of sensors that are collectively or individually designed to detect one or more parameters of the aqueous medium in which the biomass may grow. Non-limiting examples of parameters of an aqueous medium include pH, oxidation reduction potential (ORP), total dissolved solids (TDS), water hardness, temperature, conductivity, salinity, chlorine concentration, carbon dioxide (CO₂) concentration, ammonia concentration, dissolved oxygen concentration, zeta potential, and/or algae cell density. In some embodiments at least eight, at least ten, at least twelve, or all of these parameters are measured using the sensor array 100. The parameters measured by an individual sensor array may vary based on its placement in the biomass processing system 112. For example, a sensor array 100 in a growth tank may measure different parameters from a sensor array 100 location in or near a lysing process. In some embodiments, the sensor array 100 includes at least four sensors, at least six sensor, or at least eight sensors. For example, in some configurations, a sensor array 100 can be configured to measure at least the following four parameters: pH, ORP, TDS, and temperature. In other configurations, a sensor array 100 can be configured to measure at least the following five parameters, pH, ORP, TDS, zeta potential, and temperature. In other configurations, a sensor array 100 can be configured to measure at least the following five parameters, pH, ORP, cell density, and CO₂ concentration.

Various sensors can be incorporated into the sensor array 100 to measure parameters of the aqueous medium in which the biomass may grow. Some sensor arrays 100 can include at least four sensors, at least six sensors, or at least eight sensors. Non-liming examples of sensors include a pH sensor (or pH electrode), ORP sensor (or ORP electrode), a conductivity sensor, a temperature sensor, a chlorine analyzer, a fermenter control, a zeta potential sensor, and a stream current sensor. Various embodiments include at least five of these sensors, at least six of these sensors, at least seven of these sensors, at least eight of these sensors, or all of these sensors along with additional sensors.

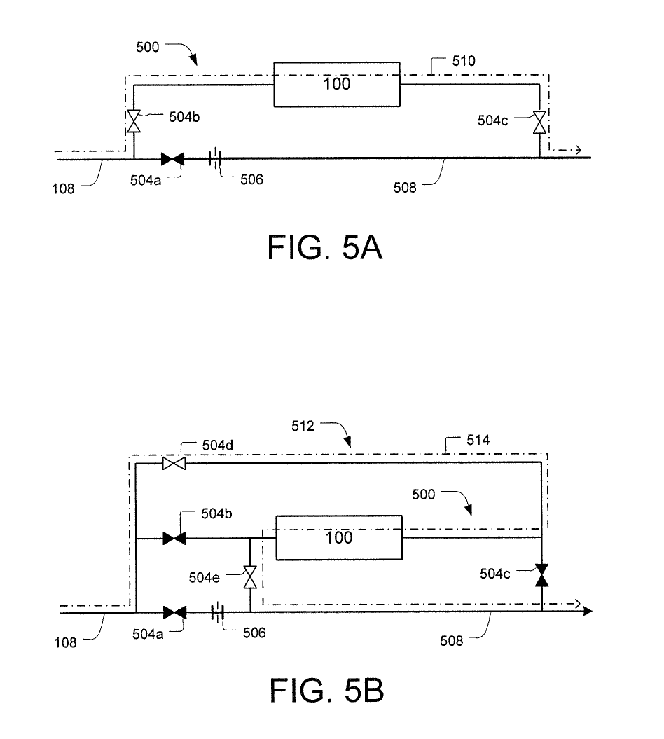

When measured, these parameters can be communicated to a controller 106 via a communication link 110. The communication link 110 can include any known or future developed wired or wireless communication link. In some embodiments, the controller 106 can be part of a Supervisory Control and Data Acquisition (SCADA) system. The controller 106 can receive and process the measured parameters and respond by controlling various aspects of the biomass processing system. For examples, the controller 106 can control the operation of systems, power supplies, modulators, frequency generators, motors, electrode pairs, nutrient delivery systems, lysing systems, flocculation systems, photoreactors, and the like. Specific examples of such controls are described with reference to FIG. 5, below.

FIG. 2 illustrates some embodiments of a sensor array 100. As shown, the sensor array 100 can be coupled to a vessel, such as a conduit 108 in which an aqueous medium resides or flows. The sensors 200 may additionally or alternatively be coupled to various other vessels of a biomass processing system. As shown, the sensor array 100 includes a plurality of sensors 200 coupled to the conduit 108. Each of the sensors 200 can have a probe member 202 disposed within the lumen 208. When an aqueous medium is present within the lumen 208, the probe member can contact the aqueous medium and test one or more of its parameters. Each of the sensors 200 may be configured to measure at least one parameter of an aqueous medium in which a biomass may grow. The measured data can be transmitted via a cable 204 or other communication link. Similarly, the sensors 200 may be powered using the cable 204 or via an internal power source, such as a battery.

In some embodiments the sensors 200 may be mounted to the conduit 108 via a wet-tap system and may be capable of withstanding a minimum of 50 psi. In some embodiments mounting of one or more of the sensors 200 can be done with threaded taps and a threaded body probe and/or a compression nut system over a smooth body probe.

Referring still to FIG. 2, in use, solid particles in the aqueous medium may tend to foul the inner surface 206 of the lumen and the surface of the probe members 202. Biomass material and other particles can accumulate on these surfaces or grow on these surfaces. To minimize the occurrence of fouling, the probe member can include a ceramic surface, a polished metal surface, and/or a coating configured to deter bio-residue formation. The materials used to construct or coat these surfaces may be selected based on the contents of the aqueous medium (e.g., high salt or chlorine content, etc.) Moreover, these surfaces may be selected from non-toxic materials that will not adversely impact the biomass feedstock. Additionally, the conduit 108, vessel, or a housing that supports the sensor array 100 can be configured to support a high flow rate through the sensor array 100 to reduce the likelihood of probe member fouling.

To improve measurement reliability, it may be useful to provide a fresh sample of aqueous medium at the probe members 202. Accordingly, in some embodiments, the positioning of the probe members 202 on the inner surface 206 of the lumen 208 can be arranged on different radial and longitudinal positions along the conduit 108 or other vessel. This arrangement can space the probe members 202 apart to prevent flow disruptions caused by one probe member to substantially affect the sample of aqueous medium presented to downstream probe members 202. Accordingly, FIG. 2 illustrates probe members 202 spaced longitudinally (left to right as shown) along the length of the conduit 202. The illustrated probe member configuration is also axially varied within the conduit, with some probe members 202 being placed on opposite sides of the conduit 108. In some embodiments, probe members may be spaced relative to each other to resist the creation of turbulent flow within the conduit 108.

Another means of improving measurement reliability can include disposing one or more flow directors within the lumen 208. Flow directors can include protrusions or depressions formed on or in the inner surface 206 of the lumen 208. These protrusions or depressions can modify and/or direct the flow of fluid through the lumen to increase the likelihood that a fresh sample of aqueous medium is present to each probe member 202. Flow directors can be configured to induce straight, spiraled, or turbulent fluid flow. For example, FIG. 2 illustrates a spiraling flow director 210 that can induce spiraled flow within the lumen 208. The spiraling flow directors 210 can be protrusions or depressions within the lumen 208. The spiraling flow directors 210 can be formed as continuous or periodic channels, recesses, veins, fins, or other suitable structures. In some embodiments, spiraling flow directors 210 can be useful when the probe members 202 are disposed substantially at or near the same radial position about the conduit 108. In these configurations, the spiraling flow directors 210 can introduce fresh samples to the series of probe members 202.

Some embodiments include a movable and dynamic flow director, which can be adjusted to control the flow within the conduit 108. Moreover, control over the flow condition inside a conduit 108 can be provided locally or remotely using a controller, such as that shown in FIG. 1. Some embodiments may be structure to be used as “indication only” of the dynamic flow condition inside a spool piece.

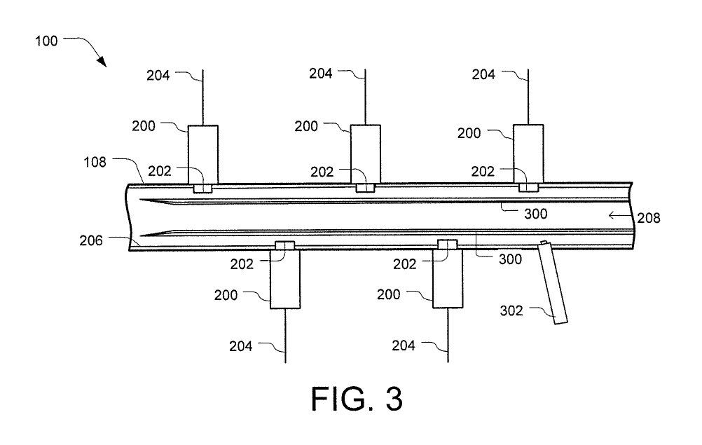

FIG. 3 illustrates a representative example of a straightening flow director 300. The straightening flow director can include protrusions or depressions within the lumen 208 that are formed as continuous or periodic channels, recesses, veins, fins, or other suitable structures. Similarly, flow directors designed to produce turbulence can be formed as continuous or periodic protrusions or depressions in the form of bumps, channels, recesses, veins, fins, or other suitable structures. In some embodiments, straightening flow directors 300 can be useful when the probe members 202 are disposed at varying radial positions about the conduit 108. In these configurations, the straightening flow directors 300 can introduce fresh samples to the series of probe members 202.

As further shown in FIG. 3, the sensor array 100 may include one or more injectors 302 coupled to the conduit 108 and configured to direct a blast of fluid towards a probe member 202. An injector can be provided for each probe member 202, or one injector 302 may be configured to clean multiple probe members 202. The injectors 302 may inject various fluid, including a liquid or gas, such as argon or CO₂.

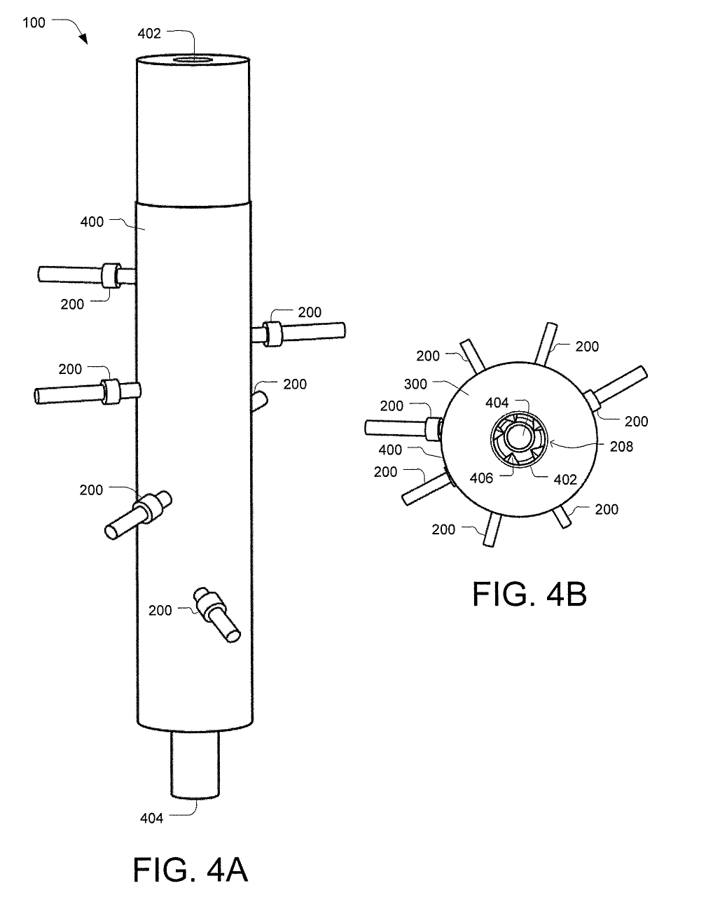

FIG. 4A illustrates a perspective view of sensor array 100, which includes a housing 400, or spool piece, that is coupled to seven separate sensors 200 (all seven are visible in FIG. 4B). The housing 400 includes an inlet 402 and an outlet 404. A lumen (208 in FIG. 4B) similar to that of FIGS. 2 and 3 can extend between the inlet 402 and the outlet 404. Similarly, the probe members (not shown) of the sensors can be disposed within the lumen of the housing where they may be exposed to an aqueous medium.

As shown, the sensors 200 can be located at varying radial positions about the housing 400. In some embodiments, the sensors can be placed in a helical pattern about the housing 400, the varying radial positions and/or helical pattern can place the probe elements (not shown) apart from one another and in a non-direct fluid path.

FIG. 4B illustrates a top view of the sensor array 100 of FIG. 4A. As shown, the lumen 208 of the housing 400 can include one or more flow directors 406.

Reference will now be made to FIGS. 5A and 5B, which illustrate a flow diagram of fluid through a biomass processing system that includes a sensor array 100. As shown, the biomass processing system can include one or more conduits through which an aqueous medium can be transported. A sensor loop 500 can be coupled to a primary flow line 508 of the biomass processing system to permit fluid to be directed from the primary flow line 508 through the sensor array 100. Disposing the sensor array 100 within a sensor loop 500 can permit the sensor array 100 to be bypassed for cleaning, easy replacement, probe member calibration, or when otherwise needed.

In some embodiments, one or more flow control devices can be incorporated into the primary flow line 508 and/or the sensor loop 500 to permit the selective deviation of flow from the primary flow line 508 through the sensor loop 500. The flow control devices can be valves 504a-504c (504 collectively) coupled to the primary flow line 508 and/or the sensor loop 500. For example, flow can be directed through the illustrated sensor loop 500 when valve 504a is closed (for illustration, valves are shown in solid black when closed) and valves 504b and 504c are opened (for illustration, valves are shown in white when open). In such instances, fluid can follow through the sensor loop 500 along the flow path 510, shown in broken lines.

In some embodiments, the flow control device can include an orifice flow restrictor (OFR) 506 which may be structured to divert a percentage (e.g., 10% to 50% or 15% to 30%) of the flow within the primary flow line 508 to the sensor loop 500.

FIG. 5B illustrates a modification to the biomass processing system of FIG. 5A, according to some embodiments, which includes the addition of a flowback loop 512. The flowback loop 512 can be used to provide back flushing or chemical cleaning through the sensor array 100 in the reverse direction, causing particle build up caused during use in the “normal” direction to be flushed out. The addition of the flowback loop 512 can include the addition of valves 504d and 504e. To operate the flowback loop 512, valves 504a, 504b, and 504 are closed and valves 504d and 504e are opened. This allows fluid to flow from the primary line 508 through the flowback loop along fluid path 514, which is shown in broken lines. In some embodiments, all or just some of the sensors can function properly during the use of the flowback loop 514, permitting continuous use of the sensor array 100 during flushing operations.

In some configurations, flushing operations through the flowback loop 512 may be routine, periodic, or on an as-needed basis. The duration of flushing operations may be based on speed of medium passing through the sensor array 100 and types of particulates in solution. Some embodiments comprise at least one flow meter (not shown) attached to the system that is used in conjunction with the biomass processing system to control pump output and log process volumes. The flow meter can assist in determining flush period duration. In some embodiments downstream probe members may tend to foul faster than upstream ones, therefore special care may be utilized for cleaning those probe members. In some embodiments, water, chemical, gas or other types for cleaners can be ran through the sensor loop 500 and/or flowback loop 512 to clean the sensor array 100. For example ultrasonic emissions during a high pressure rinse may be utilized in some cleaning processes. Also, some embodiments may utilize a chemical that would emulsify, via ultrasonics, oils and contaminants to assist in the cleaning of probe members 202.

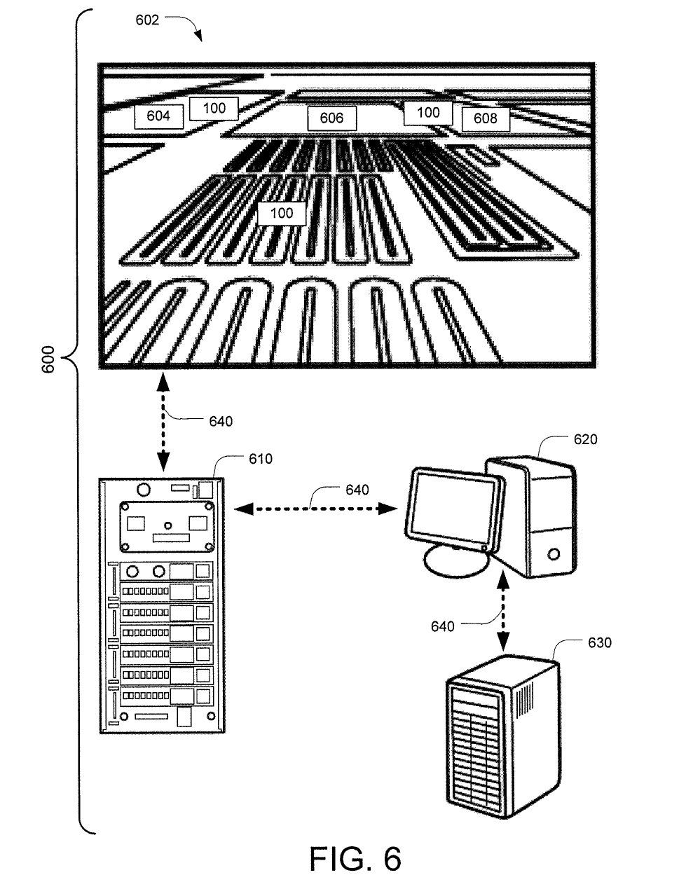

Reference will now be made to FIG. 6, which illustrates a more particular biomass processing system 600. The system includes a biomass growth and harvesting plant (“plant”) 602 that includes various systems for growing and harvesting biomass feedstock. The plant 602 can include various sub-systems, including, for example, a growth sub-system 606, a flocculation reactor sub-system 604, and a lysing reactor sub-system 608. Each of these sub-systems can include one or more sensor arrays 100, as shown. These sub-systems can be electronically coupled via a communication link 640 to one or more controllers 610, such as a programmable logic controller (PLC) and/or other computer system. The controller 610 can be electronically coupled to one or more workstations, such as a human machine interface (HMI) 620. The HMI 620 can be electronically coupled to one or more databases 630.

In operation, the controller 640 can receive measurements from sensor arrays 100 in the plant 602. Based on these measurements, the controller 610 can adjust the plant processes to optimize biomass growth and harvesting. The controller 610 can be configured with pre-programmed logic that enables it to respond to input sensor measurements and output various commands. The controller's logic may be overridden or changed by the HMI 620, through which a human user can monitor and adjust the plant processes. The HMI 620 can assist or perform manual or automatic system monitoring, data acquisition, perimeters setup, alarm control, and/or sensor calibrations. The HMI 620 and/or the database 630 can perform runtime logging, data storage, graphical reporting of system performance and/or additional information to be used for research and development.

The combination of the sensor arrays 100, the controller 610, and HMI 620 can be referred to as a SCADA system. In operation with the plant 602, the SCADA system can utilize nutrient process feedback from the sensor arrays 100 in the growth sub-system 606 to automatically or semi-automatically control the growth sub-system 606, including biomass circulation, nutrient infusions, and growth monitoring.

The SCADA system can also automatically or semi-automatically control at least some of the functions of a flocculation reactor sub-system 604 and a lysing reactor sub-system 608. For example, in some configurations, a sensor array 100 installed either or both upstream and downstream sides of the flocculation reactor sub-system 604 and the lysing reactor sub-system 608 for monitoring and calibration of the power and flow delivery to these systems. In response to measurements from the sensor arrays 100, the SCADA system can modify pulse frequency, amplitude and/or flow rates for optimal flocculation based on a predefined series of process maps.

Additionally, the SCADA system can use sensor arrays 100 as sampling and analytical tools and for point gathering measurements. An example of this would be to characterize the composition of feed water in open or closed photobioreactors, ponds or raceways in various stages of growth, maintenance, and operations. The SCADA system can also be deployed and connected to remote telemetry or local indications of water chemistry and algae culture. The SCADA system can also enable lysimiter testing and separate affects testing to be accomplished remotely and unmanned, with the capability of both static and dynamic change in state scenarios.

In some embodiments, the SCADA system utilized one parameter measured using one sensor to confirm the calibration of other sensor. For example, data from a pH sensor can be used to confirm the calibration of an alkalinity sensor/detector. Similarly a temperature sensor may be used to confirm the calibration of a flow sensor, since temperature may change with increased flow rates. Also, a dissolved oxygen sensor may be used to confirm the calibration of an ORP sensor, or vice versa.

Some embodiments of the SCADA system provide real-time reading of sensors with sampling taken on point of change. Some embodiments comprise sampling readings on time intervals, such as 50 or 100 millisecond intervals. For example, some embodiments comprise probes which read conductivity every 5 milliseconds. As a non-limiting example, if an average of 100 milliseconds is used, a total of 20 sample points may be averaged into a single data point. This can reduce database size and false readings.

Some embodiments of the SCADA system can utilize separate algorithms, which run parallel to the SCADA system processes and which are designed to predict probe changes and monitor for irregular probe behavior. Some embodiments comprise a fault flag set via software or hardware to alert an operator to evaluate, correct and clear the fault. Some embodiments are structured to stream all information to a database server for evaluation and graphical presentation.

From the foregoing, it will be seen that a biomass growth and processing systems is provided that include a sensor array configured to measure parameters of an aqueous medium containing a biomass. Using the measured parameters, the individual processes of the biomass growth and processing systems may be automatically or semi-automatically optimized to.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A sensor array comprising:
a conduit having a lumen extending between an inlet and an outlet of the conduit; and
a plurality of sensors coupled to the conduit, each of the sensors having a probe member disposed within the lumen, each of the sensors being configured to measure at least one parameter of an aqueous medium containing a biomass.

2. The sensor array of claim 1, wherein the plurality of sensors includes at least four sensors, each sensor measuring at least one parameter of the aqueous medium that is not measured by another sensor.

3. The sensor array of claim 1, wherein the plurality of sensors includes three or more of a pH sensor, an ORP sensor, a conductivity sensor, a temperature sensor, a chlorine analyzer, a fermenter control, a zeta potential sensor, and a stream current sensor.

4. The sensor array of claim 1, wherein the at least one parameter of the aqueous medium includes pH, OPR, TDS, conductivity, temperature, salinity, chlorine, ammonia, dissolved oxygen, zeta potential, and biomass cell density.

5. The sensor array of claim 1, wherein the plurality of sensors are located at varying radial positions about the conduit.

6. The sensor array of claim 5, further comprising one or more straightening flow directors disposed on an inner wall of the lumen.

7. The sensor array of claim 1, further comprising one or more spiraling flow directors disposed on an inner wall of the lumen.

8. The sensor array of claim 7, wherein the plurality of sensors are located at substantially the same radial position about the conduit.

9. The sensor array of claim 1, further comprising one or more injectors coupled to the conduit and configured to direct a blast of fluid towards the probe member of a sensor of the plurality of sensors.

10. The sensor array of claim 1, wherein the probe member includes one of a ceramic surface, a polished metal surface, and a coating configured to deter bio-residue formation.

11. A biomass processing system comprising:
a vessel configured to retain an aqueous medium containing a biomass;
a sensor array in fluid communication with the vessel, the sensor array having a plurality of sensors, each of the sensors having a probe member disposed within the vessel, each of the sensors being configured to measure at least one parameter of the aqueous medium containing a biomass; and
a controller in electronic communication with the sensor array and configured to adjust one or more processes of the biomass processing system in response to sensor data received from the sensor array.

12. The biomass processing system of claim 11, wherein the plurality of sensors includes at least four sensors, each sensor measuring at least one parameter of an aqueous medium that is not measured by another sensor.

13. The biomass processing system of claim 11 wherein the at least one parameter of an aqueous medium includes pH, OPR, TDS, conductivity, temperature, salinity, chlorine, ammonia, dissolved oxygen, zeta potential, and biomass cell density.

14. The biomass processing system of claim 11, wherein the vessel includes a conduit, the sensor array being coupled to the conduit.

15. The biomass processing system of claim 14, further comprising a bypass loop in fluid communication with the sensor array, and further comprising one or more valves configured to direct the aqueous medium through the bypass loop and to reverse the direction of fluid flow through the sensor array.

16. The biomass processing system of claim 11, wherein the vessel is one of a bioreactor, an algae growth pond, a fluid path of an algae harvesting system.

17. The biomass processing system of claim 11, wherein the controller is a programmable logic controller, and further comprising a human machine interface in electronic communication with the programmable logic controller.

18. A sensor array comprising:
a housing;
a lumen extending between an inlet and an outlet of said housing; and
a four or more sensors coupled to the housing, each of the sensors having a probe member disposed within the lumen, each of the sensors being configured to measure at least one parameter of an aqueous medium containing a biomass, each sensor being configured to measure at least one parameter of an aqueous medium that is not measured by another sensor, wherein the at least one parameter of an aqueous medium includes pH, OPR, TDS, conductivity, temperature, salinity, chlorine, ammonia, dissolved oxygen, zeta potential, and biomass cell density.

19. The sensor array of claim 18, further comprising one or more injectors coupled to the conduit and configured to direct a blast of fluid towards a probe member.

20. The sensor array of claim 18, further comprising one or more flow directors disposed on an inner wall of the lumen.
